(19) United States
(12) Patent Application Publication
(10) Pub. No.: US 2025/0332331 A1
(43) Pub. Date: Oct. 30, 2025

(54) MANIFOLDS FOR DIALYSIS APPLICATIONS AND RELATED SYSTEMS AND RELATED METHODS

(71) Applicant: MOZARC MEDICAL US LLC, Minneapolis, MN (US)

(72) Inventor: Stefano Mercati, Mirandola (IT)

(21) Appl. No.: 19/188,838

(22) Filed: Apr. 24, 2025

Related U.S. Application Data
(60) Provisional application No. 63/638,725, filed on Apr. 25, 2024.

(51) Int. Cl. A61M 1/28

(52) U.S. Cl. CPC A61M 1/28; A61M 2202/0014; A61M 2202/04; A61M 2205/0216; A61M 2205/0233; A61M 2205/025; A61M 2205/3327; A61M 2205/3331; A61M 2207/00

(57) ABSTRACT

A dialysis system includes a container for holding a dialysis fluid; and a manifold, including: a first layer having a first surface and a second surface opposite the first surface, where the second surface has a first groove formed thereon, where the first groove extends between a first inlet and a first outlet, a second layer having a third surface and a fourth surface opposite the third surface, where the third surface of the second layer is adjacent the second surface of the first layer, where the second layer includes a through-hole extending between the third surface and the fourth surface, and a third layer having a fifth surface and a sixth surface opposite the fifth surface, where the fourth surface of the second layer is adjacent the fifth surface of the third layer, where the fifth surface includes a second groove formed thereon, where the second groove extends between a second inlet and a second outlet, where the first outlet is in fluid communication with the through-hole, and the second inlet is in fluid communication with the through-hole, thereby to provide a first flow path, through the manifold, between the first inlet and the second outlet, where the container is in fluid communication with the first inlet of the manifold.

### CROSS REFERENCE

This application claims the benefit of and priority to U.S. Provisional Patent Application No. 63/638,725 filed Apr. 25, 2024, the entire content of each of which is incorporated by reference herein.

### FIELD

The present disclosure is directed to a manifold, such as for a dialysis machine, as well as a related system and a related method.

### BACKGROUND

A peritoneal dialysis machine flows a dialysis fluid (e.g., a dialysate) from a container (e.g., a dialysis bag) into a patient's peritoneal cavity, and then flows the fluid (e.g., the dialysate with waste products) out of the patient's peritoneal cavity into another container (e.g., a drainage bag).

### SUMMARY

A manifold includes a first layer having a first surface and a second surface opposite the first surface. The second surface has a first groove formed thereon. The first groove extends between a first inlet and a first outlet. A second layer of the manifold has a third surface and a fourth surface opposite the third surface. The third surface of the second layer is adjacent the second surface of the first layer. The second layer includes a through-hole extending between the third surface and the fourth surface. A third layer of the manifold has a fifth surface and a sixth surface opposite the fifth surface. The fourth surface of the second layer is adjacent the fifth surface of the third layer. The fifth surface includes a second groove formed thereon. The second groove extends between a second inlet and a second outlet. The first outlet is in fluid communication with the through-hole. The second inlet is in fluid communication with the through-hole. Thus a first flow path is provided through the manifold, between the first inlet and the second outlet.

A dialysis system includes a container configured to hold a dialysis fluid, and a manifold, and the container is in fluid communication with the first inlet of the manifold.

The container is configured to hold a fluid used in peritoneal dialysis. In some embodiments, the container is configured to hold a fluid used in hemodialysis.

At least one of the first layer and the third layer includes a metal material.

The metal material may include at least one of stainless steel, aluminum, titanium, cobalt-chrome alloy, and combinations thereof.

At least one of the first layer and the third layer may include a polymeric material.

At least one of the first layer and the third layer may include an elastomeric material.

At least one of the first layer and the third layer may include a glass material.

At least one of the first layer and the third layer may include a ceramic material.

The system may include at least one valve. The at least one valve is configured to regulate flow through the first flow path.

The at least one valve comprises a first valve. The first valve may be disposed within a first opening located in the first layer, the third layer, or both the first layer and the third layer. The first valve may further be disposed within a second opening located in the second layer.

The system further may include at least one sensor. The at least one sensor may be configured to sense a characteristic of a fluid flowing through the first flow path.

The at least one sensor may include a first sensor. At least one of the first layer and the third layer may include a first opening. The first sensor may be disposed within the first opening.

The system further may include a first sensor. At least one of the first layer and the third layer may include a first opening. The second layer may include a second opening. The first sensor may be disposed within the first opening and the second opening.

The first groove may include a curved groove section.

The second groove may include a curved groove section.

The second layer may include a metal material.

The metal material may include at least one of stainless steel, aluminum, titanium, cobalt-chrome alloy, and combinations thereof.

The second layer may include a polymeric material.

The second layer may include an elastomeric material.

The second layer may include a glass material.

The second layer may include a ceramic material.

The second layer may include a first sublayer, a second sublayer, and a third sublayer. The second sublayer may be between the first sublayer and the third sublayer. Each of the first sublayer and the third sublayer may include an elastomeric material. The second sublayer may include a metal material.

A dialysis system may include a container configured to hold a dialysis fluid, and a manifold. The container may be in fluid communication with the first through-hole.

A method of performing dialysis may include obtaining a container with a dialysis fluid and a manifold, and flowing the dialysis fluid from the container through the first flow path.

The dialysis fluid may flow from the container through the first flow path into a body of a patient undergoing peritoneal dialysis.

The method may include removing the dialysis fluid from the body of the patient.

A method of manufacturing a manifold may include providing a first layer, a second layer, and a third layer. The first layer may have a first surface and a second surface opposite the first surface, the second surface including a first groove formed thereon, the first groove extending between a first inlet and a first outlet. The second layer may have a third surface and a fourth surface opposite the third surface, the third surface of the second layer adjacent the second surface of the first layer, the second layer including a through-hole extending between the third surface and the fourth surface. The third layer may have a fifth surface and a sixth surface opposite the fifth surface, the fourth surface of the second layer adjacent the fifth surface of the third layer, the fifth surface including a second groove formed thereon, the second groove extending between a second inlet and a second outlet. The method may include positioning the first layer, the second layer, and the third layer, such that the first outlet is in fluid communication with the through-hole, and the second inlet is in fluid communication with the through-hole, thereby to provide a first flow path between the first inlet and the second outlet.

### BRIEF DESCRIPTION OF THE DRAWINGS

This section refers to the drawings that form a part of this disclosure, and which illustrate some of the embodiments of structure, materials, and/or methods described herein.

FIG. 1 is a schematic top view of a manifold, in accordance with some embodiments.

FIG. 2 is a cross-sectional view of the manifold of FIG. 1, taken along line II-II and looking in the direction of the arrows, in accordance with some embodiments.

FIG. 3 is a cross-sectional view of another embodiment of a manifold.

### DETAILED DESCRIPTION

Due to the increased complexity of pneumatic, hydraulic, and electrical systems used with and incorporated into biomedical devices, such as dialysis devices, there exists a need for modular structure that efficiently integrates these systems into the devices.

To meet such a need, the present invention provides a structure, for example in the form of a modular, multilayer valve manifold system. The structure includes pathways, as described in detail below, which integrate pneumatic, hydraulic, and/or electronic systems, while still being compact and easy to manufacture.

In some embodiments, as further described below and as shown in the figures, the structure includes an arrangement of multiple layers, which include one or more of the pathways. These layers facilitate the regulation, direction, and isolation of fluid flow, while accommodating media of different types, pressures, and temperatures. The layers and their attachment to one another prevent leaks between the layers. The structure provides these advantages while providing a reduced installation footprint over a traditional single-block manifold valve system.

In certain embodiments of the invention, the structure includes one or more pathways. The pathways may be used to combine and/or separate a fluid, such as a liquid and/or gas. One or more of the pathways may be used for wiring. One or more of the pathways may be used for pneumatics. One or more of the pathways may be grooves. The pathways may pneumatic pathways, pathways for fluid flow, or electrical wiring pathways.

Some embodiments provide a manifold. The manifold may be used in any or all of a medical system, machine, and/or method. The manifold may be used in any or all of a dialysis system, machine, and/or method. The dialysis system, machine, and/or method may be used in peritoneal dialysis. The dialysis system, machine, and/or method may be used in hemodialysis. The manifold may be used in a dialysis system, machine, and/or method in another type of dialysis. The manifold may be used in a system, machine, and/or method in another type of medical treatment. The manifold may be used in a system, machine, and/or method in application outside of medical treatment.

In some embodiments, the manifold includes at least one layer, as further described. The manifold may include more than one layer. The manifold may include two layer. The manifold may include three layers. The manifold may include four layers. The manifold may include five layers. The manifold may include six layers. The manifold may include more than six layers.

In some embodiments, at least some of the layers of the manifold contact one another. The manifold may include a first layer having a first surface and a second surface opposite the first surface. The second surface may include a first groove formed thereon. The first groove may extend between a first inlet and a first outlet. The first inlet may be formed through the first surface, and may be fluid communication with the first groove. Because the grooves or pathways are formed on surfaces of the layers, the grooves or pathways are easy to manufacture, while still able to provide a complex pneumatic or fluid flow path, or complex electrical wiring path.

The manifold may include the second layer having a third surface and a fourth surface opposite the third surface. The second layer may include a through-hole extending between the third surface and the fourth surface.

The first layer and the second layer may be positioned relative to one another, thereby to provide a flow path between the first layer and the second layer. The third surface of the second layer may be adjacent the second surface of the first layer. The through-hole in the second layer may be aligned with either the inlet or the outlet of the first layer.

The manifold may include a third layer having a fifth surface and a sixth surface opposite the fifth surface. The fifth surface may include a second groove formed thereon. The second groove may extend between a second inlet and a second outlet. The second outlet may be formed in the sixth surface, and may be in fluid communication with the second groove.

The second layer and the third layer may be positioned relative to one another, thereby to provide a flow path between the second layer and the third layer. The fourth surface of the second layer may be adjacent the fifth surface of the third layer. The through-hole in the second layer may be aligned with either the inlet or the outlet of the third layer.

The first outlet of the first layer may be in fluid communication with the through-hole of the second layer. The second inlet of the third layer may be in fluid communication with the through-hole of the second layer. The arrangement of the first layer, the second layer, and the third layer may provide a first flow path, through the manifold, between the first inlet of the first layer and the second outlet of the third layer.

In some embodiments, the system, method, or machine includes a container configured to hold a fluid. The container may be a dialysis bag. The container may be something other than a dialysis bag. The container may be configured to hold a fluid used in peritoneal dialysis. The container may be configured to hold a fluid used in hemodialysis. The container may be configured to hold a fluid used in another medical treatment or procedure.

In some embodiments, the container is in fluid communication with the first inlet of the manifold.

In some embodiments, at least one of the first layer, the second layer, and/or the third layer includes a metal material. The metal material may include at least one of stainless steel, aluminum, titanium, cobalt-chrome alloy, and combinations thereof.

At least one of the first layer, the second layer, and/or the third layer may include a polymeric material.

At least one of the first layer, the second layer, and/or the third layer may include an elastomeric material.

At least one of the first layer, the second layer, and/or the third layer may include a glass material.

At least one of the first layer, the second layer, and/or the third layer may include a ceramic material.

The system, machine, and/or method may include at least one valve. The at least one valve may be configured to regulate flow through the manifold. The at least one valve may be configured to regulate flow through the first flow path of the manifold.

At least one of the first layer and the third layer may include a first opening, and the valve may be disposed within the first opening.

At least one of the first layer and the third layer may include a first opening, the second layer may include a second opening, and the valve may be disposed within the first opening and the second opening.

The system may include at least one sensor. The sensor may be configured to sense a characteristic of a fluid flowing through the manifold. The sensor may be configured to sense a characteristic of a fluid flowing through the first flow path of the manifold. The sensor may be a transducer.

At least one of the first layer and the third layer may include a first opening, and the sensor may be disposed within the first opening.

At least one of the first layer and the third layer may include a first opening, the second layer may include a second opening, and the sensor may be disposed within the first opening and the second opening.

In some embodiments, one or more of the grooves may include a curved groove section. One or more of the grooves may include a straight groove section. One or more of the grooves may include both curved and straight groove sections.

One or more of the grooves may have a square cross section. One or more of the grooves may have a rectangular cross section. One or more of the grooves may have a triangular cross section. One or more of the grooves may have a semicircular cross section. One or more of the grooves may have an oval cross section. One or more of the grooves may have a polygonal cross section. One or more of the grooves may have cross section that may be a combination of two or more of the described cross sections.

The second layer may include at least two sublayers. The second layer may include a first sublayer, a second sublayer, and a third sublayer. The second sublayer may be between the first sublayer and the third sublayer. The first sublayer and the third sublayer may be more resilient than the second sublayer. The second sublayer may be more rigid than the first sublayer and the third sublayer. In some embodiments, each of the first sublayer and the third sublayer may include an elastomeric material, and the second sublayer may include a metal material. In some embodiments, each of the first sublayer and the third sublayer may include an elastomeric material, and the second sublayer may include a polymeric material. In some embodiments, each of the first sublayer and the third sublayer may include an elastomeric material, and the second sublayer may include a glass material. In some embodiments, each of the first sublayer and the third sublayer may include an elastomeric material, and the second sublayer may include a ceramic material.

Two or more of the layers and/or sublayers may be joined to one another in various ways. Two or more of the layers and/or sublayers may be adhered to one another with an adhesive. Two or more of the layers and/or sublayers may be welded to one another. Two or more of the layers and/or sublayers may be brazed to one another. Two or more of the layers and/or sublayers may be fastened to one another, such as with bolts, screws, and/or other fasteners. Two or more of the layers and/or sublayers may be clipped to one another, such as with one or more clips. The method of joining may be selected to prevent leaking of fluids between the layers of the device.

The one or more grooves may be machined in the layers and/or sublayers. The one or more grooves may be etched in the layers and/or sublayers.

Any or all of the first layer, the second layer, and/or the third layer may include two or more sublayers.

One or more of the layers may include more than one groove. One or more of the layers may include more than one groove.

A method may include obtaining a container with a fluid, and a manifold, as described. The method may include flowing the fluid from the container through the first flow path. The container may be a dialysis bag. The container may be something other than a dialysis bag. The fluid may be a dialysis fluid. The dialysis fluid may be a dialysate. The fluid may be something other than a dialysis fluid.

The flowing may include flowing the fluid from the container through the manifold, into the body of the patient undergoing a medical treatment. The medical treatment may be dialysis. The dialysis may be peritoneal dialysis. The method may include removing the fluid from the body of the patient. The removed fluid may be the dialysate and a waste product. Thus, the method may be a method of performing dialysis. Some embodiments provide a method of manufacturing the manifold. The method may include providing a first layer, a second layer, and a third layer, as described.

The first layer may have a first surface and a second surface opposite the first surface. The second surface may include a first groove formed thereon. The first groove may extend between a first inlet and a first outlet.

The second layer may have a third surface and a fourth surface opposite the third surface. The second layer may include a through-hole extending between the third surface and the fourth surface.

The third layer may have a fifth surface and a sixth surface opposite the fifth surface. The fifth surface may include a second groove formed thereon. The second groove may extend between a second inlet and a second outlet.

The method may include positioning, locating, and/or orienting the first layer, the second layer, and the third layer, such that the first outlet of the first layer may be in fluid communication with the through-hole of the second layer, and the second inlet of the third layer may be in fluid communication with the through-hole of the second layer, thereby to provide a first flow path between the first inlet and the second outlet.

The layers may be positioned such that third surface of the second layer may be adjacent the second surface of the first layer. The layers may be positioned such that the fourth surface of the second layer may be adjacent the fifth surface of the third layer.

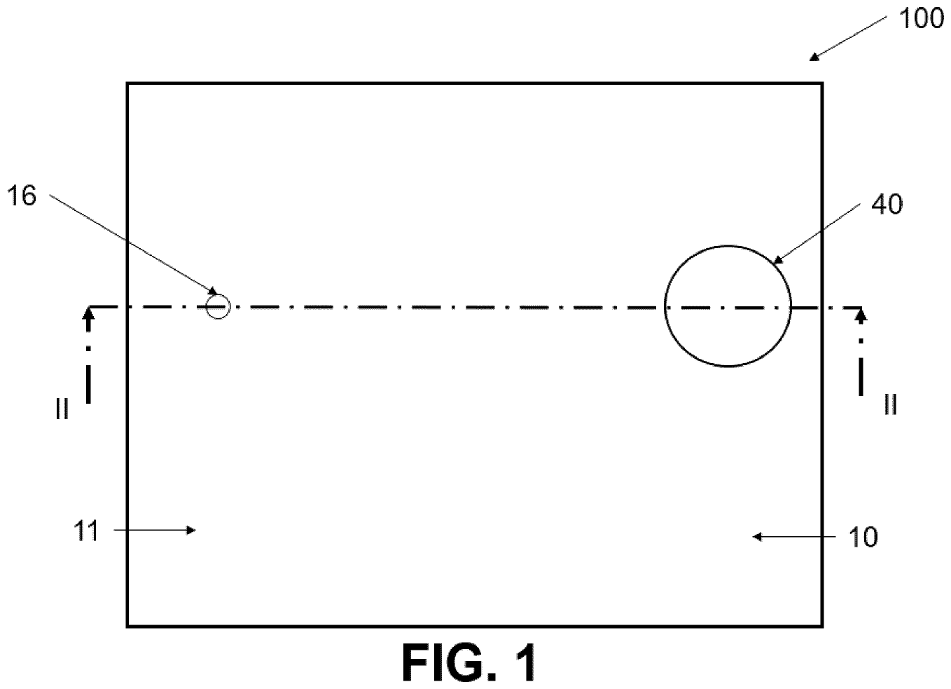

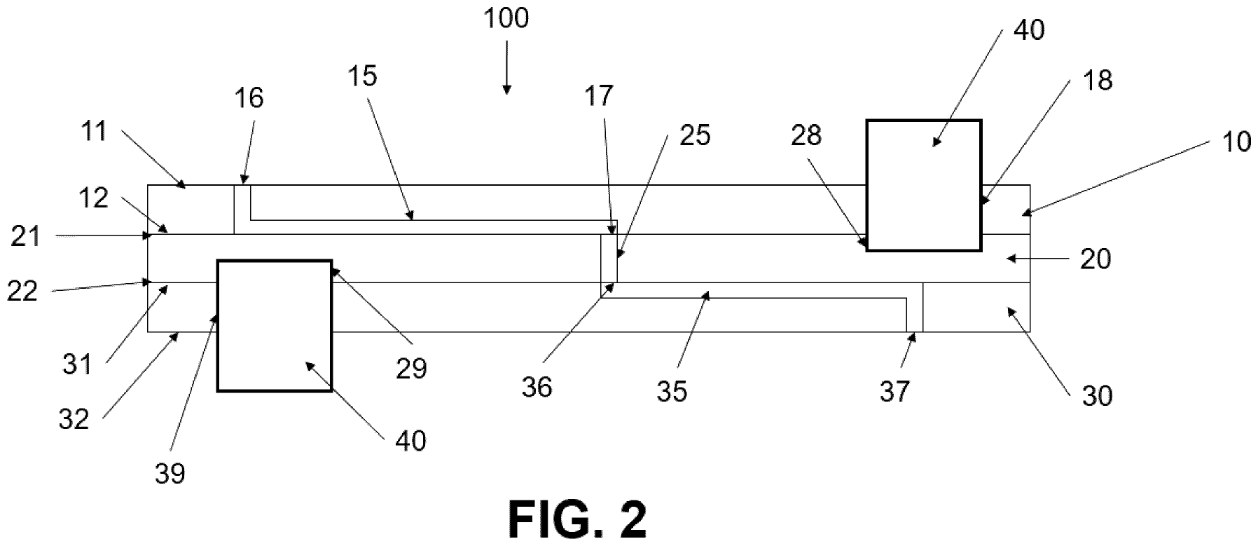

With reference to the drawings, FIG. 1 is a schematic top view of a manifold, and FIG. 2 is a cross-sectional view of the manifold of FIG. 1, taken along line II-II and looking in the direction of the arrows, in accordance with some embodiments. As the figures show, a manifold 100 may include a first layer 10, a second layer 20, and a third layer 30. In some embodiments, one or more, or all, of the first layer 10, the second layer 20, and/or the third layer 30 may be in accordance with the descriptions of the first layer, the second layer, and/or the third layer, as set forth above. As the figure shows, the manifold 100 may include components 40. The components 40 may be in accordance with the sensor, valve, and/or transducer described above. The manifold 100 may include one component 40. The manifold 100 may include more than two components 40. In some embodiments, the manifold 100 omits the components 40.

As the figures show, in some embodiments, one component 40 may be disposed within an opening 18 in the first layer 10 and an opening 28 in the second layer 20. As the figures show, another component 40 may be disposed in an opening 39 in the third layer 30 and an opening 29 in the second layer 20.

The first layer 10 may include a first surface 11, and a second surface 12 opposite the first surface 11. The first layer 10 may include a first groove 15 formed in the second surface 12. As the figure illustrates, the first groove 15 may include a first inlet 16 and a first outlet 17. The first inlet 16 may be formed in the first surface 11, to thereby provide a fluid flow path from outside of the first layer 10 into the first groove 15.

The second layer 20 may include a third surface 21, and a fourth surface 22 opposite the third surface 21. The second layer 20 may include a through-hole 25, which extends between the third surface 21 and the fourth surface 22. As the figure shows, the third surface 21 of the second layer 20 may be positioned adjacent the second surface 12 of the first layer 10. The through-hole 25 may be aligned with the first outlet 17, as shown.

The third layer 30 may include a fifth surface 31, and a sixth surface 32 opposite the fifth surface 31. The fifth surface 31 may include a second groove 35 formed in the fifth surface 31. As the figures illustrate, the second groove 35 may include a second inlet 36 and a second outlet 37. The second outlet 37 may be formed in the sixth surface 32, to thereby provide a fluid flow path from outside of the third layer 30 into the second groove 35.

As the figure shows, the fourth surface 22 of the second layer 20 may be positioned adjacent the fifth surface 31 of the third layer 30. The through-hole 25 may be aligned with the second inlet 36 of the third layer 30, as shown. Thus, as described, there may be a flow path through the manifold 50 from the first inlet 16, through the first groove 15, through first outlet 17, through the through-hole 25, through the second inlet 36, through the second groove 35, through the second outlet 37. It is understood that although the above description describes flow from the first inlet 16 to the second outlet 37, fluid may be flowed otherwise through the manifold 50. For example, in some embodiments, fluid may be flowed from the second outlet 37 to the first inlet 16.

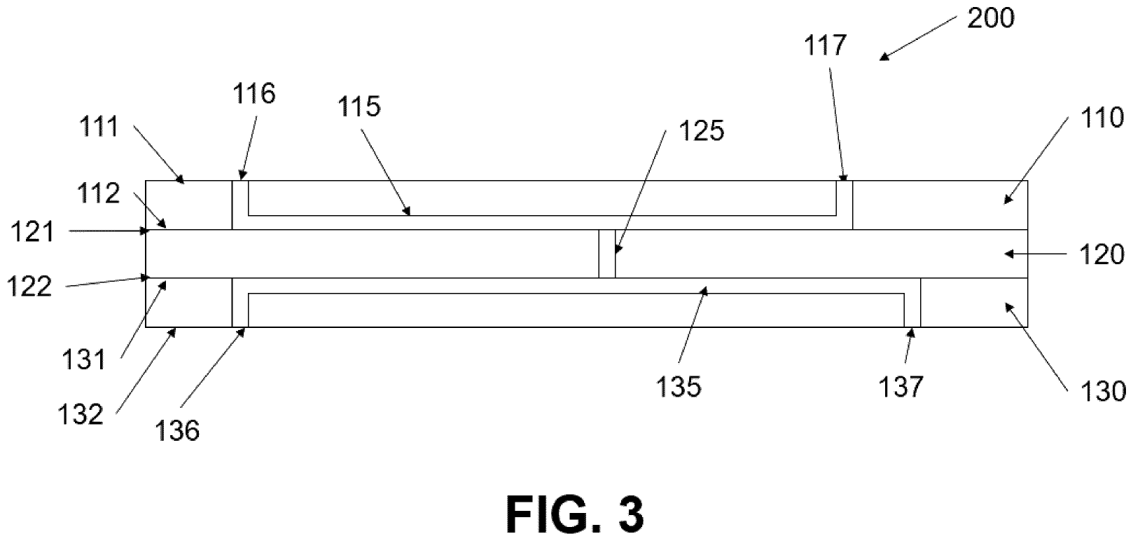

FIG. 3 shows an alternate embodiment of the manifold, in accordance with some embodiments of the invention. As the figure shows, a manifold 200 may include a first layer 110, a second layer 120, and a third layer 130. In some embodiments, one or more, or all, of the first layer 110, the second layer 120, and/or the third layer 130 are in accordance with the descriptions of the first layer, the second layer, and/or the third layer, as set forth above.

The first layer 110 may include a first surface 111, and a second surface 112 opposite the first surface 111. The first layer 110 may include a first groove 115 formed in the second surface 112. As the figure illustrates, the first groove 115 may include a first inlet 116 and a first outlet 117. The first inlet 116 may be formed in the first surface 111, to thereby provide a fluid flow path from outside of the first layer 110 into the first groove 115. The first outlet 117 may be formed in the first surface 111, to thereby provide a fluid flow path from outside of the first layer 110 into the first groove 115.

The second layer 120 may include a third surface 121, and a fourth surface 122 opposite the third surface 121. The second layer 120 may include a through-hole 125, which extends between the third surface 121 and the fourth surface 122. As the figure shows, the third surface 121 of the second layer 120 may be positioned adjacent the second surface 112 of the first layer 10. The through-hole 125 may be in fluid communication with the first groove 115, as shown.

The third layer 130 may include a fifth surface 131, and a sixth surface 132 opposite the fifth surface 131. The fifth surface 131 may include a second groove 135 formed in the fifth surface 131. As the figures illustrate, the second groove 135 may include a second inlet 136 and a second outlet 137. The second inlet 136 may be formed in the sixth surface 132, to thereby provide a fluid flow path from outside of the third layer 130 into the second groove 135. The second outlet 137 may be formed in the sixth surface 132, to thereby provide a fluid flow path from outside of the third layer 130 into the second groove 135.

As FIG. 3 shows, the fourth surface 122 of the second layer 120 may be positioned adjacent the fifth surface 131 of the third layer 130. The through-hole 125 may be aligned with the second groove 135 of the third layer 130, as shown. Thus, there are multiple fluid flow paths between and/or among the first inlet 116, the first outlet 117, the second inlet 136, and/or the second outlet 137, including but not limited to:
a flow path from first inlet 116, through the first groove 115, to first outlet 117, as well as in the opposite direction;
a flow path from the second inlet 136, through the second groove 135, to the second outlet 137, as well as in the opposite direction;
a flow path from the first inlet 116, through the first groove 115, through the through-hole 125, through the second groove 135, to the second inlet 136, as well as in the opposite direction;
a flow path from the first outlet 117, through the first groove 115, through the through-hole 125, through the second groove 135, to the second outlet 137, as well as in the opposite direction;
a flow path from the first inlet 116, through the first groove 115, through the through-hole 125, through the second groove 135, to the second outlet 137, as well as in the opposite direction; and/or
a flow path from the first outlet 117, through the first groove 115, through the through-hole 125, through the second groove 135, to the second inlet 136, as well as in the opposite direction.

Thus, there may be a flow path through the manifold 50 from the first inlet 16, through the first groove 15, through first outlet 17, through the through-hole 25, through the second inlet 36, through the second groove 35, through the second outlet 37. It is understood that although the above description describes flow from the first inlet 16 to the second outlet 37, there are other flow paths through the manifold 50. For example, in some embodiments, fluid may be flowed from the second outlet 37 to the first inlet 16.

Although the figures show only one groove in the first layer and the third layer, one inlet and outlet in each of the first layer and the third layer, and one through-hole in the second layer, it is understood that the layers are not limited to one groove, and, in some embodiments, one or more of the layers may include two to ten (or more) grooves, for example; the layers are not limited to one inlet and one outlet, and, in some embodiments, one or more of the layers may include two to ten or more inlets and/or outlets, for example; and/or, the layers are not limited to one through-hole, and, in some embodiments, one or more of the layers may include two to ten or more through-holes, for example.

In another embodiment, the manifold includes-multiple layers, one or more of which may be one or more of the layers described above and shown in the figures. The manifold may include one or more of the layers in a different order. In some embodiments, one or more layers of the manifold are adjacent one another, rather than stacked on one another. In some embodiments, the manifold may include at least one of the described layers, with other components.

1. A dialysis system, comprising:
a container configured to hold a dialysis fluid; and
a manifold, comprising:
a first layer having a first surface and a second surface opposite the first surface,
wherein the second surface comprises a first groove formed thereon,
wherein the first groove extends between a first inlet and a first outlet,
a second layer having a third surface and a fourth surface opposite the third surface,
wherein the third surface of the second layer is adjacent the second surface of the first layer,
wherein the second layer comprises a through-hole extending between the third surface and the fourth surface, and
a third layer having a fifth surface and a sixth surface opposite the fifth surface,
wherein the fourth surface of the second layer is adjacent the fifth surface of the third layer,
wherein the fifth surface comprises a second groove formed thereon,
wherein the second groove extends between a second inlet and a second outlet,
wherein
the first outlet is in fluid communication with the through-hole, and
the second inlet is in fluid communication with the through-hole,
thereby to provide a first flow path, through the manifold,
between the first inlet and the second outlet,
wherein the container is in fluid communication with the first inlet of the manifold.

2. The dialysis system of claim 1, wherein at least one of the first layer and the third layer comprises a metal material or polymeric material.

3. The dialysis system of claim 2, wherein the metal material comprises at least one of stainless steel, aluminum, titanium, cobalt-chrome alloy, and combinations thereof.

4. The dialysis system of claim 1, further comprising at least one valve,
wherein the at least one valve is configured to regulate flow through the first flow path.

5. The dialysis system of claim 4, wherein the at least one valve comprises a first valve,
wherein at least one of the first layer and the third layer comprises a first opening,
wherein the first valve is disposed within the first opening.

6. The dialysis system of claim 1, further comprising a first valve,
wherein at least one of the first layer and the third layer comprises a first opening,
wherein the second layer comprises a second opening,
wherein the first valve is disposed within the first opening and the second opening.

7. The dialysis system of claim 1, further comprising at least one sensor,
wherein the at least one sensor is configured to sense a characteristic of a fluid flowing through the first flow path.

8. The dialysis system of claim 7, wherein the at least one sensor comprises a first sensor,
wherein at least one of the first layer and the third layer comprises a first opening,
wherein the first sensor is disposed within the first opening.

9. The dialysis system of claim 1, further comprising a first sensor,
wherein at least one of the first layer and the third layer comprises a first opening,
wherein the second layer comprises a second opening,
wherein the first sensor is disposed within the first opening and the second opening.

10. The dialysis system of claim 1, wherein the first groove comprises a curved groove section.

11. The dialysis system of claim 1, wherein the second groove comprises a curved groove section.

12. The dialysis system of claim 1, wherein the second layer comprises a metal material.

13. The dialysis system of claim 12, wherein the metal material comprises at least one of stainless steel, aluminum, titanium, cobalt-chrome alloy, and combinations thereof.

14. The dialysis system of claim 1, wherein the second layer comprise an elastomeric material.

15. The dialysis system of claim 1, wherein the second layer comprises:
a first sublayer,
a second sublayer, and
a third sublayer,
wherein the second sublayer is between the first sublayer and the third sublayer,
wherein each of the first sublayer and the third sublayer comprises an elastomeric material,
wherein the second sublayer comprises a metal material.

16. A method of manufacturing a manifold, comprising:
obtaining a first layer, a second layer, and a third layer,
wherein the first layer has a first surface and a second surface opposite the first surface,
wherein the second surface comprises a first groove formed thereon,
wherein the first groove extends between a first inlet and a first outlet,
wherein the second layer has a third surface and a fourth surface opposite the third surface,
wherein the third surface of the second layer is adjacent the second surface of the first layer,
wherein the second layer comprises a through-hole extending between the third surface and the fourth surface,
wherein the third layer has a fifth surface and a sixth surface opposite the fifth surface,
wherein the fourth surface of the second layer is adjacent the fifth surface of the third layer,
wherein the fifth surface comprises a second groove formed thereon,
wherein the second groove extends between a second inlet and a second outlet; and
positioning the first layer, the second layer, and the third layer, such that the first outlet is in fluid communication with the through-hole, and the second inlet is in fluid communication with the through-hole, thereby to provide a first flow path between the first inlet and the second outlet.

17. The method of claim 16, further comprising:
joining the first layer, the second layer, and the third layer together.

18. A method of performing dialysis, comprising:
obtaining a container with a dialysis fluid and a manifold,
wherein the manifold comprises:
a first layer having a first surface and a second surface opposite the first surface,
wherein the second surface includes a first groove formed thereon,
wherein the first groove extends between a first inlet and a first outlet,
a second layer having a third surface and a fourth surface opposite the third surface,
wherein the third surface of the second layer is adjacent the second surface of the first layer,
wherein the second layer includes a through-hole extending between the third surface and the fourth surface, and
a third layer having a fifth surface and a sixth surface opposite the fifth surface,
wherein the fourth surface of the second layer is adjacent the fifth surface of the third layer,
wherein the fifth surface includes a second groove formed thereon,
wherein the second groove extends between a second inlet and a second outlet,
wherein
the first outlet is in fluid communication with the through-hole, and
the second inlet is in fluid communication with the through-hole,
thereby to provide a first flow path, through the manifold,
between the first inlet and the second outlet; and
flowing the dialysis fluid from the container through the first flow path.

19. The method of claim 18, wherein the flowing further comprises flowing the dialysis fluid from the container through the first flow path into a body of a patient undergoing peritoneal dialysis.

20. The method of claim 19, further comprising:
removing the dialysis fluid from the body of the patient
